(19) United States
(12) Patent Application Publication
(10) Pub. No.: US 2010/0196498 A1
(43) Pub. Date: Aug. 5, 2010

(54) Emulsion hydro therapy or milk therapy for treatment of psoriasis

(76) Inventors: Christian O. Gruhl, Dresden (DE); Ursula Gruhl, Dresden (DE)

Correspondence Address: HORST M. KASPER, 13 FOREST DRIVE, WARREN, NJ, 07059, US

(21) Appl. No.: 12/322,298

(22) Filed: Jan. 30, 2009

(51) Int. Cl. A61K 35/20; A61P 17/06

(52) U.S. Cl. 424/535

(57) ABSTRACT

It is an object of the Invention furnish a method for treatment of psoriasis at the human body for the purpose of healing this disease by way of bacteriophages. The treatment of the psoriasis patient is performed with a whole bath. The bath liquid comprises 95 percent water and 5 percent fresh milk or 100 percent fresh milk. The treatment temperature amounts to about 99 degrees Fahrenheit over the complete treatment time of 30 minutes. A glycerol ointments to be applied to the patient at the erythematous positions after termination of the bath. The treatment period is daily over 14 days.

### BACKGROUND OF THE INVENTION

1 Field of the Invention

The present invention relates to a treatment method for psoriasis.

2. Brief Description of the Background of the Invention Including Prior Art

The bacteriophages were first described in 1915. Since that time these have found increasingly applications in the medical field. The bacteriophage therapy has been increasingly successfully applied in Russia based on the lack of antibiotics. The bacteriophages serve in Germany in the medical field mostly the diagnostics for differentiating for example the vibrio cholerae biovar cholera form biovar eltor, this is called lysotyping, further possibilities based on the phagovar determination are for example for staphylococcus epidermis (Medizinisches Landesuntersuchungsamt Stuttgart) and listeria monocytogenes (Institute Pasteur, Paris). Bacteriophages are virus like corpuscular elements.

Based on the presence of bacteriophages and their possibility to be used in a lysozyme process of the bacterium, the inflicted bacterium is brought to blow up and is thereby destroyed. A precondition for this process is that the afflicted host cell either contains the genome of the bacteriophage or edge positioned extra cellular bacteriophages actively inject their DNA/RNA into the pathogenous bacteria cell.

### Surrounding Circumstances:

Psoriasis is the chronic skin disease (dermatosis) occuring predominantly with light skinned human beings and with a morbidity of 1 percent to two percent annually.

The risk of catching psoriasis is disposed between the 15th and 35th life year, as well as between the 55th and 60th life year. An inflammation is of course also caused by immuno-supressive diseases, such as post infection with HIV, or also after feverous infections with streptococcus at tonsillitis/angina and this can lead to an outbreak or manifestation of psoriasis.

The medicamentous therapy represents a further inflammatory effect such as for example with lithium and beta blocker. The selection of present-day therapies for the psoriasis falls onto the corticosteroids, photo chemotherapy with UV-A, as well as with psoralen in combination with the UVA irradiation, retinoids, cyclosporins, and methotrexate. The systemic disease of the psoriasis is characterized by a psychic and social as well as also an economic disability and handicap of the patient. Also the social integration of the patient is therewith made difficult, which leads in most cases to an intensification of the immunosuppression.

The long-term applications of the photo therapy leads to a loss in substance of the subcutaneous collagen tissue and thus to an aging and tensioning loss of the skin, and an increase of the skin cancer risk is also possible. The long-term application of the methotrexate leads to damage of the liver. The giving of retinoids has to be performed under most strict monitoring in case of women, since it can lead to different birth defects during the pregnancy.

A concrete treatment method for treating psoriasis by way of bacteriophages was described in the following:

http://www.pubmedcentral.nih.gov/picrender.fcgi?tool=
pmcentrez&artid=408469&blobtype=pdf

with the title:
“CTLA4Ig-mediated blockade of T-cell costimulation in patients with psoriasis vulgaris”
Portions of this work have appeared in abstract form (1997.
Received for publication Nov. 20, 1998, and accepted in revised form Mar. 15, 1999.
Engagement of the B7 family of molecules on antigen-presenting cells with their T cell-associated ligands,
CD28 and CD152 (cytotoxic T lymphocyte-associated antigen-4 [CTLA-4]), provides a pivotal costimulatory signal in T-cell activation. We investigated the role of the CD28/CD 152 pathway in psoriasis in a 26-week, phase I, open-label dose-escalation study. The importance of this pathway in the generation of humoral immune responses to T cell-dependent neoantigens, bacteriophage [ ]X174 and keyhole limpet hemocyanin, was also evaluated. Forty-three patients with stable psoriasis vulgaris received 4 infusions of the soluble chimeric protein CTLA4Ig (BMS-188667). Forty-six percent of all study patients achieved a 50% or greater sustained improvement in clinical disease activity, with progressively greater effects observed in the highest-dosing cohorts. Improvement in these patients was associated with quantitative reduction in epidermal hyperplasia, which correlated with quantitative reduction in skin-infiltrating T cells. No markedly increased rate of intralesional T-cell apoptosis was identified, suggesting that the decreased number of lesional T cells was probably likely attributable to an inhibition of T-cell proliferation, T-cell recruitment, and/or apoptosis of antigen-specific T cells at extralesional sites. Altered antibody responses to T cell-dependent neoantigens were observed, but immunologic tolerance to these antigens was not demonstrated.

This study illustrates the importance of the CD28/CD152 pathway in the pathogenesis of psoriasis and suggests a potential therapeutic use for this novel immunomodulatory approach in an array of T cell-mediated diseases. J. Clin. Invest. 103:1243-1252 (1999).

### SUMMARY OF THE INVENTION

### 1. PURPOSE OF THE INVENTION

It is an object of the present Invention to furnish a method for treatment of psoriasis at the human body for healing this disease by way of bacteriophages.

It is another object of the invention to heal psoriatic plaques and red scaly patches on the skin of a human being.

It is yet a further purpose of the invention to furnish an organic treatment method for psoriasis.

These and other objects and advantages of the present invention will become evident from the description which follows.

### 2. BRIEF DESCRIPTION OF THE INVENTION

The present invention provides and the advantages of the treatment method according to the present invention comprise that one can use only natural and allergen free materials as well as one can work with the smallest technical expenditure. No complicated and cost intensive monitoring is any longer necessary for the patient. Also the presence of a doctor is not necessary. The applications of the treatment according to the present invention are completely without side effects. The treatment agents are very cost advantageous. The concept of treatment can also be incorporated into the private household region and adapts itself thus to the everyday life of the patient.

It is a further advantage that the patient does not need special schooling in order to perform himself or herself the treatment, a simple physical understanding on the side of the patient is desirable but is not a condition. A specific biochemical or physical pre-processing of the emulsion is not necessary, since simply only the part ratio of water and milk or only the treatment with 100 percent milk is performed. A milk emulsion is defined in the context of the present application as a mixture of from 100 to 5 volume percent milk with from 0 to 95 volume percent water. A milk emulsion containing 100 volume percent milk represents an emulsion of whole milk. A milk emulsion containing 5 volume percent milk represents an emulsion containing 5 volume percent milk and 95 volume percent water. An active milk emulsion can contain a mixture of from 100 to 60 volume percent milk with from 0 to 40 volume percent water. An economy milk emulsion can contain a mixture of from 60 to 5 volume percent milk with from 40 to 95 volume percent water.

A method for treatment of psoriasis comprises preparing a milk emulsion; bringing the milk emulsion to a temperature of from 95 to 113 degrees Fahrenheit and forming a warm milk emulsion, contacting psoriatic plaque delimited skin positions in contact with the warm milk emulsion, agitating the warm milk emulsion over the psoriatic plaque delimited skin positions, interacting the warm milk emulsion with the psoriatic plaque delimited skin positions, and removing the milk emulsion from the psoriatic plaque delimited skin positions.

The emulsion hydro therapy for treatment of psoriasis is performed in case of a whole body application such that the patient obtains a whole bath with an application time of from 15 to 45 minutes in an emulsion, wherein the emulsion comprises the components of 0.1 percent to maximum 95 percent water and of from about five percent to 99.9 percent of fresh milk if possible still warm from the animal body, and wherein the emulsion has a treatment temperature of at least 95 degrees Fahrenheit to 113 degrees Fahrenheit and wherein the erythematous sharply and with scale delimited skin positions or psoriatic plaques and red scaly patches on the skin are continuously brought in contact with the new emulsion by motion of the emulsion, and wherein this treatment procedure is performed daily over a treatment duration of from 10 days to 30 days.

In many cases it is sufficient that the patient obtains a partial bath at a partial body application in a dish, a bucket or other container with an application time of from 15 to 45 minutes in an emulsion, wherein the emulsion comprises the components of 0.1 to maximum 95 cent water and from five percent to 99.9 percent fresh milk possibly still warm from the animal body, and wherein the emulsion has a treatment temperature of at least 95 degrees Fahrenheit to 1 13 degrees Fahrenheit, and wherein the erythematous sharply and with scale delimited skin positions continuously are brought in contact with new emulsion through motion of the emulsion and wherein the treatment procedure is daily performed over a treatment period of from 10 days to 30 days.

A milk therapy can be furnished for treatment of psoriasis, wherein the patient obtains a whole bath with an application time of from 15 minutes to 45 minutes in 100 percent fresh milk possibly still warm from the animal body, wherein the treatment temperature of the fresh milk during the treatment time amounts to between 95 degrees Fahrenheit and 113 degrees Fahrenheit, and wherein the erythematous sharply and with scale delimited skin positions are continuously brought in contact with new fresh milk by motion of the fresh milk, and wherein this treatment procedure is performed daily over a treatment period of 10 days to 30 days.

A patient can obtain a partial bath in a dish, bucket, or other container in a partial body application with an application time of from 15 to 45 minutes in fresh milk possibly still warm from the animal body, wherein the treatment temperature of the fresh milk during the treatment time amounts to between 95 degrees Fahrenheit and 113 degrees Fahrenheit, and wherein the erythematous sharply and with scale delimited skin positions are continuously brought in contact with the new fresh milk through motion of the fresh milk, and wherein this treatment procedure is performed daily over a treatment period of from 10 days to 30 days.

The erythematous sharply and with scale delimited skin positions or psoriatic plaques or red scaly patches are after the bath covered with a pure glycerol ointment or with a glycerol chamomile ointment. The skin positions affected with the psoriasis are covered with pure glycerol ointment or with a glycerol chamomile ointment. This advances additionally the healing process.

The therapy can be performed as a whole body application or with individual body parts in a container adapted to the body shape, wherein a circulation of the emulsion or milk is furnished with uniform temperature. The use of emulsion or, respectively, the employed amount of milk can be substantially reduced.

The liquid level depends on the well-being of the patient, if however the liquid level was set according to the New York heart association then the height of the liquid level can lie up to a maximum at the height level of the heart. Therefore sick persons with heart and circulation problems can also obtain the treatment according to the present invention.

The applications are also performed as a face affusion and/or head affusion with emulsion or with milk, wherein advantageously a continuous circulation of the emulsion or, respectively, the milk is performed by way of a pump with simultaneous temperature control.

The applications are performed also as whole body affusion with emulsion or with milk. A whole body affusion is also possible, however this will certainly remain an exception of the invention treatment.

A tuberculin test is performed at the patient prior to the beginning of the therapy.

The performing of a tuberculin test at the patients prior to the therapy assures the patient against a possible tuberculosis disease caused by the therapy. In case of a presence of tuberculosis pathogens in the fresh milk, there would exist no danger in case of a presence of defense forces of the patient. In case the tuberculin test would be negative, then the patient can receive a corresponding vaccination prior to the therapy.

The invention is in the following explained in more detail by way of embodiment examples.

### EMBODIMENT EXAMPLE 1

The application chamber is preheated and the previously disinfected bath container, for example a tub as a full bath, is filled with an emulsion amount necessary for the treatment of 285 liter water and of 15 liter of fresh milk possibly warm from the animal body. The ratio of water to milk amounts here to 95 percent to 5 percent. The treatment temperature of the emulsion should be held at about 99 degrees Fahrenheit during the complete treatment time of 30 minutes. The patient should move in the emulsion during the treatment bath, since this maintains the convection of the water and thereby enables a uniform remaining temperature in the bath container, as well as a new contact of the bacteriophages in the emulsion with the skin dermatosis in the bath container. In addition, also body positions are wetted with fresh emulsion through motions of the patient, which positions otherwise do not come in contact emulsion based on the resting in the tub. The motion of the emulsion can also be performed by a pump system with integrated heating. The temperature control of the emulsion is thereby also alleviated. After termination of the bath after 30 minutes, a pure glycerol ointment or a glycerol chamomile ointment is applied at the erythematous positions of the patient, which ointment shall be completely drawn back. This treatment method is to be daily continued over a treatment period of 14 days.

### EMBODIMENT EXAMPLE 2

In this treatment method of psoriasis patients there is changed only the treatment agents relative to the embodiment example 1. Pure milk is employed here instead of the emulsion. The use of foremilk (colostrum) is possible also in special cases. All other conditions as the treatment time, temperature and so on are performed or, respectively, applied in the same way as described in embodiment example 1.

### EMBODIMENT EXAMPLE 3

The use of a container adapted to the human body, similar to a space suit, or only to individual body parts such as arms and legs adapted containers are furnished for reducing the emulsion or milk volume. By employing a pump coupled with a heating system, temperature control of the emulsion or milk is easily made possible. The treatment conditions correspond however in detail to those of the embodiment example 1.

### EMBODIMENT EXAMPLE 4

For treatment of the head or, respectively, face region of psoriasis patients preferably head affusions or, respectively, face affusions are employed. The patient is disposed for this purpose in a seated position slightly inclined toward the rear. The head is here angled in the neck. A capturing dish for the emulsion or milk is disposed under the head. Advantageously, a capturing dish is employed, which is furnished with a pump, coupled with a heating system, which allows the emulsion or milk to exit over a swivelable exit system like an affusion onto the head. All skin positions afflicted with psoriasis are to be cast on continuously through the swivelable exit system with emulsion or milk through a helper. Since a change of the positions to be cast on is necessary in case of larger concerned skin positions, the treatment time is thereby lengthened to beyond 30 minutes. The ratio of water to milk as an emulsion and also the treatment temperature correspond to the values from the embodiment example 1.

1. A method for treatment of psoriasis comprising the following steps:
preparing a milk emulsion;
bringing the milk emulsion to a temperature of from 95 to 1 13 degrees Fahrenheit and forming a warm milk emulsion;
contacting psoriatic plaque delimited skin positions in contact with the warm milk emulsion;
agitating the warm milk emulsion over the psoriatic plaque delimited skin positions;
interacting the warm milk emulsion with the psoriatic plaque delimited skin positions;
removing the milk emulsion from the psoriatic plaque delimited skin positions.

2. The method according to claim 1,
wherein in case of a whole body application a patient obtains a whole bath with an application time of from 15 to 45 minutes in an emulsion, wherein the emulsion comprises components of 0.1 percent to maximum 95 percent water and of from about five percent to 99.9 percent of fresh milk, and wherein the emulsion has a treatment temperature of at least 95 degrees Fahrenheit to 1 13 degrees Fahrenheit and wherein the psoriatic plaque delimited skin positions are continuously brought in contact with a new emulsion by motion of the emulsion, and wherein a treatment procedure is performed daily over a treatment duration of from 10 days to 30 days.

3. The method according to claim 1,
wherein a patient obtains a partial bath at a partial body application in a dish, a bucket or other container with an application time of from 15 to 45 minutes in an emulsion, wherein the emulsion comprises components of 0.1 to a maximum of 95 percent water and from five percent to 99.9 percent fresh milk, and wherein the emulsion has a treatment temperature of at least 95 degrees Fahrenheit to 113 degrees Fahrenheit, and wherein the psoriatic plaque delimited skin positions continuously are brought in contact with new emulsion through motion of the emulsion and wherein a treatment procedure is daily performed over a treatment period of from 10 days to 30 days.

4. The method according to claim 1,
wherein a patient obtains a whole bath with an application time of from 15 minutes to 45 minutes in 100 percent fresh milk, wherein a treatment temperature of the fresh milk during a treatment time amounts to between 95 degrees Fahrenheit and 113 degrees Fahrenheit, and wherein the psoriatic plaque delimited skin positions are continuously brought in contact with new fresh milk by motion of the fresh milk, and wherein this treatment procedure is performed daily over a treatment period of 10 days to 30 days.

5. The method according to claim 1,
wherein a patient obtains a partial bath in a dish, bucket, or other container in a partial body application with an application time of from 15 to 45 minutes in fresh milk, wherein a treatment temperature of the fresh milk during a treatment time amounts to between 95 degrees Fahrenheit and 113 degrees Fahrenheit, and wherein the psoriatic plaque delimited skin positions are continuously brought in contact with new fresh milk through motion of the fresh milk, and wherein a treatment procedure is performed daily over a treatment period of from 10 days to 30 days.

6. The method according to claim 1,
wherein the psoriatic plaque delimited skin positions are after the bath covered with a pure glycerol ointment or with a glycerol chamomile ointment.

7. The method according to claim 1,
wherein the contacting is performed as a whole body application or with individual body parts in a container adapted to the body shape, wherein a circulation of the emulsion or milk is furnished with uniform temperature.

8. The method according to claim 1,
wherein a liquid level depends on the well-being of the patient, or the liquid level is set according to the New York heart association where the height of the liquid level can lie up to a maximum at the height level of the heart.

9. The method according to claim 1,
wherein applications are also performed as a face affusion or head affusion with emulsion or with milk, wherein advantageously a continuous circulation of the emulsion or, respectively, the milk is performed by way of a pump with simultaneous temperature control.

10. The method according to claim 1,
wherein applications are performed also as whole body affusion with emulsion or with milk.

11. The method according to claim 1, further comprising
performing a tuberculin test at a patient prior to the beginning of treatment; and
wherein the milk is still warm from the animal body.

12. A method employing emulsion hydro therapy for treatment of psoriasis wherein in case of a whole body application a patient obtains a whole bath with an application time of from 15 to 45 minutes in an emulsion, wherein the emulsion comprises components of 0.1 percent to a maximum of 95 percent water and of from about five percent to 99.9 percent of fresh milk if possible still warm from an animal body, and wherein the emulsion has a treatment temperature of at least 95 degrees Fahrenheit to 113 degrees Fahrenheit and wherein erythematous sharply and with scale delimited skin positions are continuously brought in contact with a new emulsion by motion of the emulsion, and wherein a treatment procedure is performed daily over a treatment duration of from 10 days to 30 days.

13. The method according to claim 12 wherein the patient obtains a partial bath at a partial body application in a dish, a bucket or other container with an application time of from 15 to 45 minutes in an emulsion, wherein the emulsion comprises the components of 0.1 to maximum of 95 percent water and from five percent to 99.9 percent fresh milk possibly still warm from an animal body, and wherein the emulsion has a treatment temperature of at least 95 degrees Fahrenheit to 113 degrees Fahrenheit, and wherein erythematous sharply and with scale delimited skin positions continuously are brought in contact with new emulsion through motion of the emulsion and wherein the treatment procedure is daily performed over a treatment period of from 10 days to 30 days.

14. A method employing milk therapy for treatment of psoriasis, wherein a patient obtains a whole bath with an application time of from 15 minutes to 45 minutes in 100 percent fresh milk possibly still warm from an animal body, wherein a treatment temperature of the fresh milk during a treatment time amounts to between 95 degrees Fahrenheit and 113 degrees Fahrenheit, and wherein erythematous sharply and with scale delimited skin positions are continuously brought in contact with new fresh milk by motion of the fresh milk, and wherein a treatment procedure is performed daily over a treatment period of 10 days to 30 days.

15. The method according to claim 14, wherein the patient obtains a partial bath in a dish, bucket, or other container in a partial body application with an application time of from 15 to 45 minutes in fresh milk possibly still warm from the animal body, wherein the treatment temperature of the fresh milk during the treatment time amounts to between 95 degrees Fahrenheit and 113 degrees Fahrenheit, and wherein the erythematous sharply and with scale delimited skin positions are continuously brought in contact with the new fresh milk through motion of the fresh milk, and wherein this treatment procedure is performed daily over a treatment period of from 10 days to 30 days.

16. The method according to claim 14,
wherein the erythematous sharp and with scale delimited skin positions are after the bath covered with a pure glycerol ointment or with a glycerol chamomile ointment.

17. The method according to claim 14,
wherein the treatment is performed as a whole body application or with individual body parts in a container adapted to the body shape, wherein a circulation of the emulsion or milk is furnished with uniform temperature.

18. The method according to claim 14,
wherein a liquid level depends on the well-being of the patient, if however the liquid level was set according to a New York heart association then a height of the liquid level can lie up to a maximum at a height level of a heart.

19. The method according to claim 14,
wherein the applications are also performed as a face affusion and/or head affusion with emulsion or with milk, wherein advantageously a continuous circulation of the emulsion or, respectively, the milk is performed by way of a pump with simultaneous temperature control.

20. The method according to claim 14,
wherein the applications are performed also as whole body affusion with emulsion or with milk; and
wherein a tuberculin test is performed at the patient prior to a beginning of treatment.
